(19) United States
(12) Patent Application Publication
(10) Pub. No.: US 2003/0170187 A1
(43) Pub. Date: Sep. 11, 2003

(54) Skin treatments containing nano-sized vitamin K

(76) Inventor: Alfred Marchal, Waterloo (BE)

Correspondence Address: Hillary W. Hawkins, Williams Mullen, 1021 East Cary Street, Richmond, VA, 23219, US

(21) Appl. No.: 10/378,570

(22) Filed: Mar. 3, 2003

Related U.S. Application Data
(60) Provisional application No. 60/361,234, filed on Mar. 1, 2002.

(51) Int. Cl. A61K 7/135; A61K 9/127

(52) U.S. Cl. 424/62

(57) ABSTRACT

This invention relates to skin treatments containing nano-sized vitamin K. Specifically, the invention relates to a skin treatment containing nano-sized vitamin K for the use in improving the aesthetic aspects of the skin.

### CROSS REFERENCE TO RELATED APPLICATION

This application claims the benefit of U.S. Provisional Application No. 60/361,234 filed in the United States Patent Office on Mar. 1, 2002.

### BACKGROUND OF THE INVENTION

1. Technical Field of the Invention

This invention relates to skin treatments containing nano-sized vitamin K. Specifically, the invention relates to a skin treatment containing nano-sized vitamin K for the use in improving the aesthetic aspects of the skin. 2. Discussions of the Related Art

The use of vitamin K for various skin care treatments is known in the art. U.S. Pat. No. 5,510,391 describes a method for treating blood vessel disorders of the skin using non-nano-sized (“conventional”) vitamin K. Such disorders include actinic and iatrogenic purpura, lentigines, telangiectasias of the face, spider angiomas and spider veins of the face.

The present invention describes a more effective and efficient way to use vitamin K in treating the skin. The use of nano-sized vitamin K provides for enhanced penetration through the skin, and therefore, the current treatment has a much quicker response time.

### SUMMARY OF THE INVENTION

The present invention is skin treatment containing nano-sized vitamin K. The treatment may be in the form of a cream, gel, lotion and/or liquid.

The skin treatment described herein is used for the improvement of various aesthetic aspects of the skin. These improvements include the reduction of the reddened, black and/or blue appearance of the skin.

It is an object of the present invention to provide a method of treating various skin disorders by using nano-sized vitamin K.

It is another object of the present invention to provide a topical skin treatment comprising nano-sized vitamin K.

It is yet another object of the present invention to provide a topical skin treatment composition that has controlled penetration, long-term efficiency, protection against oxidative degradation and regulation of the skin.

### DETAILED DESCRIPTION

In a preferred embodiment, the nano-sized vitamin K is present in an amount equal to at least about 2% by weight of the composition.

In one embodiment, the invention is a topical gel containing 5% Vitamin K, 2% of which is nano-sized. The microparticles of nano-sized vitamin K and conventional vitamin K are combined with vitamin A, vitamin C and other active cosmetic agents to produce a gel that has a positive effect on skin that is reddened or black and blue. It is for topical use and can be used around the eyes, arms and legs to effectively and quickly reduce the discoloration of the skin, and accelerate healing.

This embodiment preferably contains the following ingredients: nano-sized vitamin K, vitamin A, vitamin C, water, propylene glycol, panthenol, triethanolamine, phytonadione, lecithin, carbomer, ethoxydiglycol, phospholipids, ascorbic palmitate, retinyl palmitate, ethylenediaminetetraacetic acid (EDTA), tocopherol, acetate of tocopheryl.

In another embodiment, the invention can be used as a topical cream for use on dark circles or splotches under the eyes. This embodiment contains nano-sized vitamin K plus retinol. With regular use it improves the aesthetic aspects and provides a more youthful look. Its particular properties allow reflection of light which minimizes the transparency of the skin under the eyes.

This embodiment preferably includes the following components: water, alcohol, C₁₂-C₁₅ alkyl benzoate, caprylic capric triglycerides, parafinum liquidum, cyclomethicone, glycerine, lecithin, sodium pyrrolidone carboxylate (PCA), mica, phospholipids, barium sulfate, phytonadione, titanium dioxide, polysorbate 20, retinol, acrylate copolymer, phenoxyethanol, acrylate/C₁₀-C₃₀ alkyl acrylate crosspolymer, triethanolamine, carbomer, disodium EDTA, propyl paraben, methyl paraben, butylated hydroxytoluene (BHT) and butylated hydroxyanisole (BHA).

In a preferred embodiment, the present invention involves a dispersion system containing nano-sized vitamin K encapsulated within phospholipidic spheres. This dispersion system has excellent moisture-binding capacity. The phospholipid improves the chemical stability of the vitamin K and enhances the power of penetration through the skin.

Phospholipids are important compounds that occur in human cells. Chemically, a phosopholipid is glycerol with fatty acids of varying length and degree of unsaturation. One form of phospholipid is phosphatidylcholine.

In a preferred embodiment, the nano-sized vitamin K of the present invention is contained within a monolayer of phosphatidycholine. In one embodiment, the resulting particles are approximately 180 nanometers in diameter. The concentration of vitamin K within the encapsulated product may be up to 30%. This concentration of vitamin K is important to the final formulation. It is necessary to avoid the use of too much phospholipid in order to maintain the stability of the formula.

Some of the advantages of this nanosome encapsulation process include: protection against oxidative degradation, controlled penetration, regulation of the skin, and long-term efficiency of the vitamin K.

### EXAMPLE

For the purposes of this example, three creams with different concentrations of vitamin K were-used. Various-media were employed. The example was double blind and was applied to a sample consisting of 12 volunteers.

The study was performed in two phases with 6 persons per phase:

Group A (three males and three females)

Group B (one male and five females)

Ecchymosis was induced in each patient on 4 areas (2 on each forearm) and the efficacy of each cream was evaluated by observing the time required to reduce each case of ecchymosis. Each patient was examined every other day and pictures were taken at the same time. No other cream was applied and the patients were not allowed to take any other medication (no aspirin, no anti-inflammatory treatments).

Materials

Group A

| | |
|---|---|
| #1 | 5% conventional vitamin K cream |
| #2 | 2% nano-sized vitamin K gel |
| #3 | 0.5% nano-sized vitamin K gel |

Group B

| | |
|---|---|
| #1 | 2% nano-sized vitamin K gel |
| #2 | 5% conventional vitamin K cream |
| #3 | 0.5% nano-sized vitamin K gel |

Method

A 2 ml blood sample was taken from the ante-cubital vein of each patient and was split into four 0.5 ml subcutaneous injections in the forearms (two right, two left) to induce bruising. The injection sites were numbered 1 to 4. Sites 1 to 3 received creams 1 to 3 and site 4 remained untreated as control.

Results

Group A

| Patient #A1: F, 39 years old | | |
|---|---|---|
| Bruise reduction: | #2: | day 9 |
| | #4: | day 10 |
| | #1 & #: | day 11 |
| Patient #A2: F, 41 years old | | |
| Bruise reduction: | #2: | day 8 |
| | #1, 3 & 4: | day 11 |
| Patient #A3: M, 41 years old | | |
| Bruise reduction: | #2: | day 10 |
| | #1, 3 & 4: | day 13 |
| Patient #A4: F, 35 years old | | |
| Very fast reduction in bruises #1 & 2 | | |
| Patient #A5: M, 39 years old | | |
| Bruise reduction: | #1 & 2: | day 11 |
| | #3 & 4: | day 14 |
| Patient #A6: M, 43 years old | | |
| Bruise reduction: | #2: | day 11 |
| | #1, 3 & 4: | day 13 |

Group B

| Patient #B1: F, 44 years old | | |
|---|---|---|
| Bruise reduction: | #1 | 9 days |
| | #3: | 10 days |
| | #2 & 4: | 12 days |
| Patient #B2: F, 42 years old | | |
| Bruise reduction: | #1: | 11 days |
| | #2: | 12 days |
| | #3 & #4: | 13 days |
| Patient #B3: F, 38 years old | | |
| Bruise reduction: | #1: | 11 days |
| | #4: | 12 days |
| | #2 & #3: | 13 days |
| Patient #B4: M, 38 years old | | |
| Bruise reduction: | #1 & #3: | 10 days |
| | #2: | 12 days |
| | #4: | 13 days |
| Patient #B5: F, 38 years old | | |
| Bruise reduction: | #3: | 11 days |
| | #1 & #2: | 12 days |
| | #4: | 13 days |
| Patient #B6: F, 55 years old | | |
| Bruise reduction: | #2 & #4: | 11 days |
| | #1 & #3: | 12 days |

All the creams were well tolerated and no allergic reaction was observed.

Conclusion

Group A

Four of six cases showed a faster reduction of the bruises with cream #2 (two days sooner). The conclusion is that cream #2, which contains 2% nano-sized vitamin K gel is the most effective.

Group B

Four of six cases showed a faster reduction of the bruises with cream #1 (two days sooner). Same conclusion as Group A.

Quantitative results in days of reduction of bruises

| | | 5% | | |
|---|---|---|---|---|
| Patient | 2% nanosized | conventional | 0.5% nanosized | Untreated |
| A1 | 9 | 11 | 11 | 10 |
| A2 | 8 | 11 | 11 | 11 |
| A3 | 10 | 13 | 13 | 13 |
| A4 | 10 | 10 | 13 | 13 |
| A5 | 11 | 11 | 14 | 14 |
| A6 | 11 | 13 | 13 | 13 |
| B1 | 9 | 12 | 10 | 12 |
| B2 | 11 | 12 | 13 | 13 |
| B3 | 11 | 13 | 13 | 12 |
| B4 | 10 | 12 | 10 | 13 |
| B5 | 12 | 12 | 11 | 13 |
| B6 | 11 | 12 | 12 | 11 |
| Total | 123 | 142 | 144 | 148 |
| Averge | 10.25 | 11.83 | 12.00 | 12.33 |

Many improvements, modifications and additions will be apparent to the skilled artisan without departing from the spirit and scope of the present invention as described herein.

I claim:

1. A topical skin treatment composition comprising at least from about 2% of nano-sized vitamin K.

2. The composition of claim 1 wherein said nano-sized vitamin K is encapsulated in phospholipid spheres.

3. The composition of claim 2 wherein said phospholipid sphere is phosphatidycholine.

4. The composition of claim 1 further comprising conventional vitamin K.

5. The composition of claim 2 further comprising: vitamin A, vitamin C, water, propylene glycol, panthenol, triethanolamine, phytonadione, lecithin, carbomer, ethoxydiglycol, phospholipids, ascorbic palmitate, retinyl palmitate, EDTA, tocopherol and acetate of tocopheryl.

6. The composition of claim 5 wherein said composition is used to treat discoloration of the skin.

7. The composition of claim 2 further comprising: water, alcohol, C₁₂-C₁₅ alkyl benzoate, caprylic capric triglycerides, parafinum liquidum, cyclomethicone, glycerine, lecithin, sodium PCA, mica, phospholipids, barium sulfate, phytonadione, titanium dioxide, polysorbate 20, retinol, acrylate copolymer, phenoxyethanol, acrylate/C₁₀-C₃₀ alkyl acrylate crosspolymer, triethanolamine, carbomer, disodium EDTA, propyl paraben, methyl paraben, BHT and BHA.

8. The composition of claim 7 wherein said composition is used to minimize the transparency of the skin.

9. The composition of 1 wherein said composition is in a form selected from the group consisting of a gel, a lotion, a cream and a liquid.

10. The composition of claim 2 wherein said nano-sized vitamin K comprises at least about 30% of the phospholipid sphere.

11. A method of treating discoloration of human skin comprising:
(a) formulating a pharmaceutical composition wherein said composition contains at least about 2% by weight nano-sized vitamin K;
(b) applying said pharmaceutical composition topically to treat discoloration of the skin.

12. The method of claim 11 wherein said composition further comprises: vitamin A, vitamin C, water, propylene glycol, panthenol, triethanolamine, phytonadione, lecithin, carbomer, ethoxydiglycol, phospholipids, ascorbic palmitate, retinyl palmitate, EDTA, tocopherol and acetate of tocopheryl.

13. The method of claim 11 wherein said nano-sized vitamin K is encapsulated in phospholipid spheres.

14. The method of claim 13 wherein said phospholipid sphere is phosphatidycholine.

15. A method of minimizing the transparency of human skin comprising:
(a) formulating a pharmaceutical composition wherein said composition contains at least about 2% by weight nano-sized vitamin K;
(b) applying said pharmaceutical composition topically to treat discoloration of the skin

16. The method of claim 15 wherein said composition further comprises: water, alcohol, C₁₂-C15 alkyl benzoate, caprylic capric triglycerides, parafinum liquidum, cyclomethicone, glycerine, lecithin, sodium PCA, mica, phospholipids, barium sulfate, phytonadione, titanium dioxide, polysorbate 20, retinol, acrylate copolymer, phenoxyethanol, acrylate/C₁₀-C₃₀ alkyl acrylate crosspolymer, triethanolamine, carbomer, disodium EDTA, propyl paraben, methyl paraben, BHT and BHA.

17. The method of claim 15 wherein said nano-sized vitamin K is encapsulated in phospholipid spheres.

18. The method of claim 17 wherein said phospholipid sphere is phosphatidycholine.
